(19) United States
(12) Patent Application Publication
(10) Pub. No.: US 2025/0000697 A1
(43) Pub. Date: Jan. 2, 2025

(54) TISSUE CARRIER

(71) Applicant: Timothy W. Olsen, Rochester, MN (US)

(72) Inventor: Timothy W. Olsen, Rochester, MN (US)

(21) Appl. No.: 18/215,636

(22) Filed: Jun. 28, 2023

(51) Int. Cl. A61F 9/007

(52) U.S. Cl. CPC A61F 9/007; A61B 2017/00867; A61B 2017/00969; A61B 17/30

(57) ABSTRACT

A device includes a first band, a second band, and a joint. The first band is configured as a first ring. The second band is configured as a second ring. The joint couples the first band and the second band in stacked alignment and having a gap therebetween. The joint has a thrust surface configured to receive an applied compressive force in a direction lying substantially within a plane corresponding to at least one of the first ring or the second ring. The joint has a tension surface configured to receive an applied tensile force substantially aligned on the direction. The joint is compliant about a rotary axis lying in the plane. The joint is substantially non-compliant about an axis out of the plane.

### TECHNICAL FIELD

This document pertains generally, but not by way of limitation, to a surgical device for treating an eye disease.

### BACKGROUND

Macular degeneration is a leading cause of vision loss for people over age 50. Effective treatment remains elusive. One approach includes a transplantation procedure. Complexities associated with transplantation include difficulty in supporting and manipulating the excised tissue.

The following US documents refer variously to a surgical support structure, a tissue support structure, and a tissue graft fixture: US 2007-0179512, US 2015-0223929, US 2013-0073054, US 2017-0100283, US 2023-0157871, and U.S. Pat. No. 10,278,808.

### SUMMARY

An example of the present subject matter includes a tissue carrier. The tissue carrier is configured for retaining and stabilizing an excised segment of tissue. The tissue can be that of an eye. A tissue carrier can be configured to stabilize and carry the tissue segment to enable manipulating and placement of a graft at a selected site.

In some examples of the present subject matter, a tissue carrier includes two ring-shaped members coupled by a joint. The joint is configured as a live hinge in order to facilitate a surgical procedure. The joint can include a through-hole disposed in a pendent segment coupled to one ring of a pair of rings. The through-hole can receive a tool and provide a positive engagement that accommodates a compressive force or a tensile force and facilitates manipulation of the device.

For example, a through-hole in a pendent segment can be configured to receive a corresponding pin and clamping device to enable precise control of the position and orientation of the carrier, and thus that of the tissue carried within. Furthermore, surfaces of the carrier can be configured to receive directed forces in either a compressive or a tensile direction, which enables the user to further manipulate the placement of the tissue.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 includes a perspective view of a device, according to one example.

FIG. 2A includes a view of a device, according to one example.

FIGS. 2B and 2C include view of a joint, according to one example.

FIG. 3 includes a view of a device, according to one example.

FIG. 4 includes a view of a blank utilized in manufacturing, according to one example.

FIG. 5 includes a view of a joint, according to one example.

FIG. 6 includes a flow chart of a method, according to one example.

FIG. 7 includes a view of a device in manufacturing, according to one example.

### DETAILED DESCRIPTION

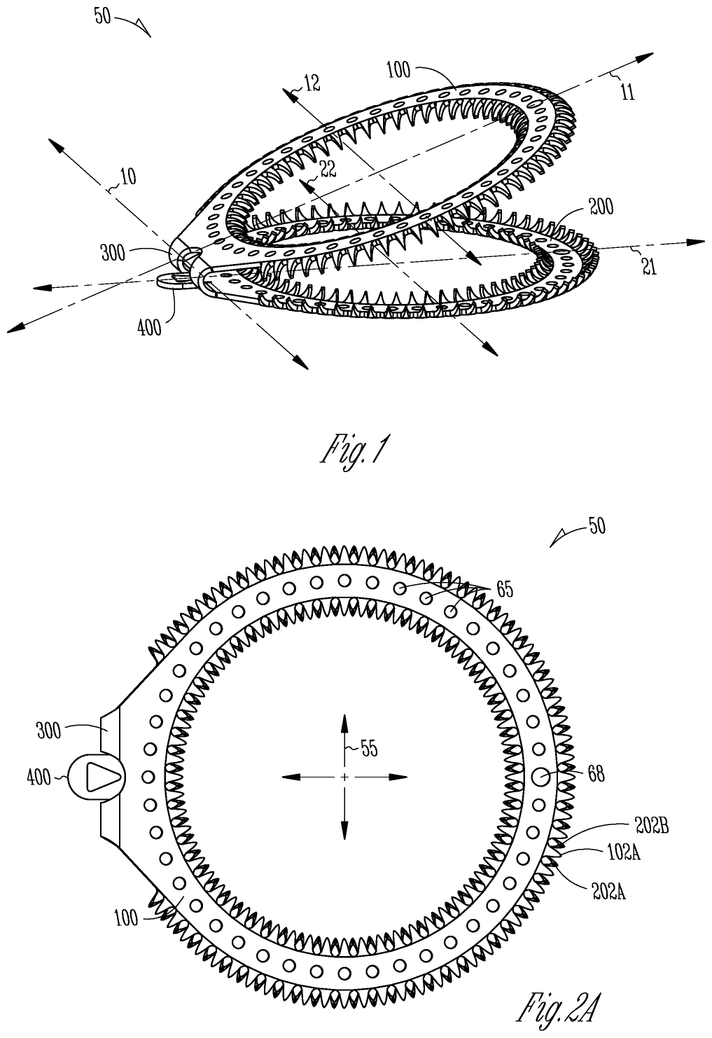

FIG. 1 illustrates device 50, according to one example. Device 50 includes first band 100, second band 200, and joint 300. Both first band 100 and second band 200 are ring-shaped elements and coupled by joint 300. In the figure, joint 300 allows for rotary motion about axis 10 as between first band 100 and second band 200. Joint 300 resists or precludes relative movement along an axis not parallel with axis 10.

First band 100 and second band 200 include a plurality of transverse holes. In addition, first band 100 and second band 200 include a plurality of teeth arranged about an interior edge of the bands and about an exterior edge of the bands. The teeth and the holes can facilitate bonding of the device with tissue. The teeth at the edges of the bands are set or deflected inward to grasp the tissue held therebetween.

Joint 300 can include a live hinge in which the material of first band 100 and second band 200 is the same as that of joint 300. A live hinge allows for relative rotational movement about the axis without resort to a hinge pin and corresponding hinge barrels. In this sense, joint 300 is integral with first band 100 and second band 200.

Axis 11 is shown to lie in the plane of first band 100 and pass through the center of joint 300, pass through the center of the ring, and pass through the ring segment distal to joint 300. Axis 12 is shown to also pass through the plane of first band 100 and pass through the center of the ring, and pass through opposing midpoints of the ring between joint 300 and the distal segment.

Axis 21 is shown to lie in the plane of second band 200 and pass through the center of joint 300, pass through the center of the ring, and pass through the ring segment distal to joint 300. Axis 22 is shown to also pass through the plane of second band 200 and pass through the center of joint 300 and pass through opposing midpoints of the ring between joint 300 and the distal segment.

Axes 11 and 12 lie in the plane of first band 100. Axes 21 and 22 lie in the plane of second band 200. First band 100 and second band 200 articulate about joint 300 at axis 10. Joint 300 precludes relative movement of first band 100 and second band 200 not aligned with rotation about axis 10.

At joint 300, device 50 includes pendent 400 extending in the plane of second band 200. Pendent 400 is disposed on one side of axis 10 and the rings of first band 100 and second band 200 are disposed on an opposing side of axis 10.

In the figure, first band 100 and second band 200 of device 50 are shown to be partially opened. That is, the plane of first band 100 and second band 200 intersect along axis 10 and the angle therebetween is acute.

When first band 100 and second band 200 are brought together, that is, substantially in parallel, the planes are substantially parallel. As such, joint 300 is compliant about a rotary axis lying in the plane and joint 300 is substantially non-compliant about an axis out of the plane.

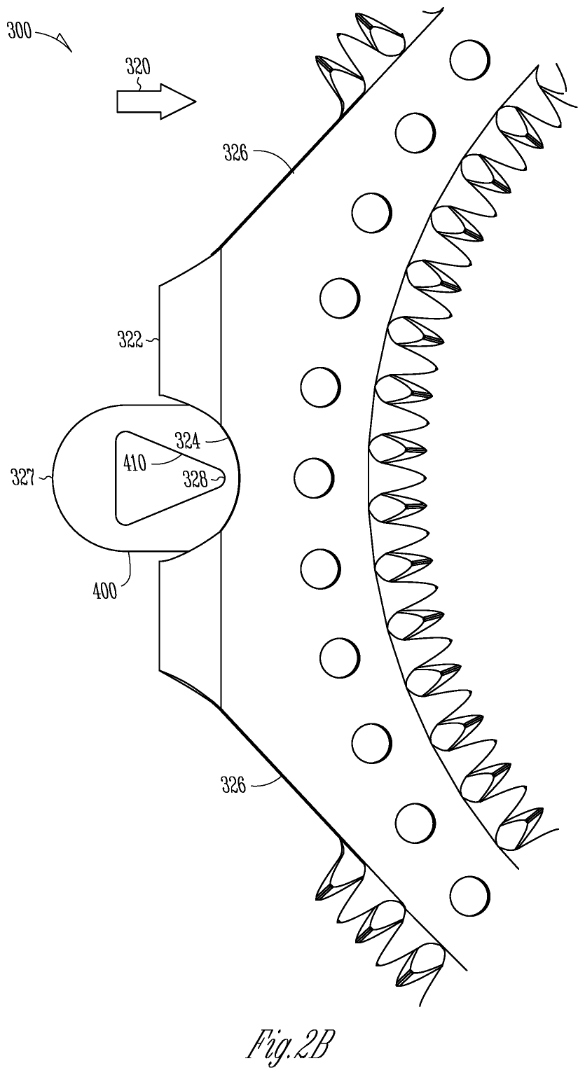

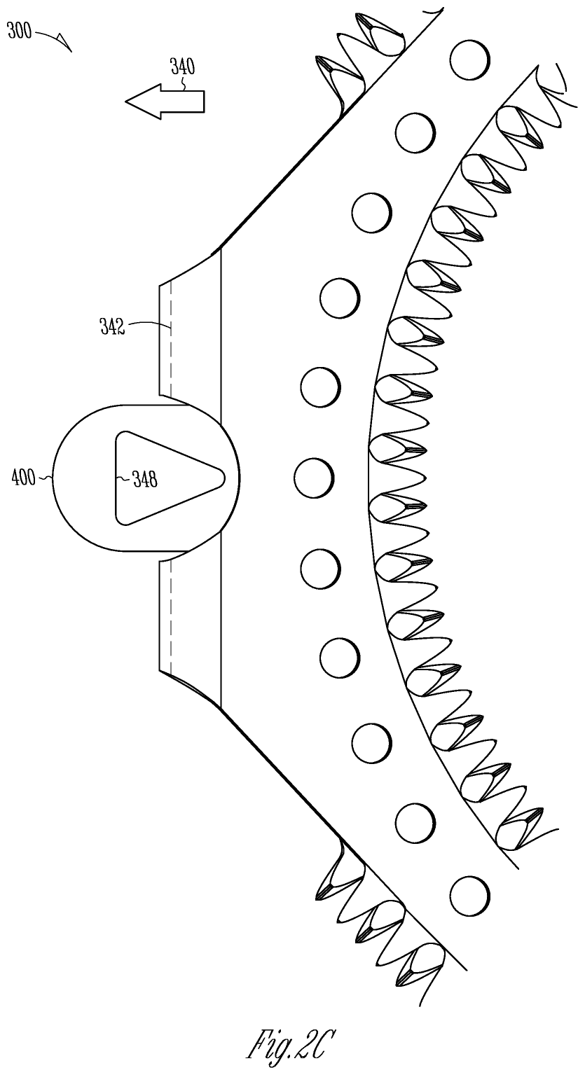

FIGS. 2A, 2B, and 2C illustrate view of device 50, according to one example, FIG. 2A includes a view of first band 100 in the foreground and second band 200 in the background and partially visible. Pendent 400 is coupled to second band 200 and is visible at a radiused cut-out of first band 100. Pendent 400, in the example shown, includes a triangular aperture.

FIG. 2A depicts a plurality of teeth arranged in a sawtooth manner about the interior and exterior edges of first band 100. The plurality of teeth on first band 100 includes tooth 102A as indicated. Second band 200 includes a plurality of teeth arranged in a sawtooth manner about the interior and exterior edges. The plurality of teeth on second band 200 includes tooth 202A and tooth 202B in the manner shown. Tooth 102A of first band 100 is aligned between tooth 202A and tooth 202B of second band 200.

As shown, first band 100 and second band 200 are in stacked alignment and substantially coaxial about center 55.

Joint 300 couples first band 100 and second band 200, as shown. Pendent 400 extends from second band 200 proximate joint 300. Aperture 68 passes through the ring of first band 100, and in some examples, passes through the ring of second band 200. Aperture 68 is shown to have a size larger than that of band holes 65, however, in other examples, the hole size of aperture 68 is smaller or the same as band holes 65.

FIG. 2B depicts an enlarged view of joint 300. In the figure, pendent 400 includes triangular aperture 410. Aperture 410 is a through-hole. In addition to a triangular shape, aperture 410 can have a square form, a round form, or a polygon form.

Device 50 can be manipulated by applying a physical force in the direction shown by arrow 320. A compressive force, applied in the direction as shown by arrow 320, can be exerted on a thrust surface of device 50. FIG. 2B illustrates some portions of device 50, proximate to joint 300, which provide a thrust surface on which a compressive force can be exerted. For example, a thrust surface can be seen at flanks 326 (on first band 100, on second band 200, or on both first band 100 and on second band 200) disposed on either side of joint 300. As another example, a thrust surface can be seen at radiused surface 322, disposed on either side of pendent 400, at joint 300. As another example, a thrust surface can be seen at cut-out portion 324 of first band 100. Cut-out portion 324 corresponds to the radius of edge 327 of pendent 400 As another example, a thrust surface can be seen at portion 328 of aperture 410. In the figure, portion 328 is proximate an apex of triangular shaped aperture 410. A compressive force exerted on one or more of these thrust surfaces can propel device 50 in the direction of the forward, or lateral plane, shown at arrow 320.

In the example shown, the internal edge of the ring of first band 100 and second band 200 includes a plurality of teeth and the external edge of the rings, in a region proximate joint 300, is devoid of teeth.

FIG. 2C, like FIG. 2B, depicts an enlarged view of joint 300. Device 50 can be manipulated by applying a physical force in the direction shown by arrow 340. A tensile force, applied in the direction of arrow 340, can be exerted on a tension surface of device 50. FIG. 2B illustrates some portions of device 50, proximate to joint 300, which provide a tension surface on which a tensile force can be exerted. For example, a tension surface can be seen at inside radius 342 disposed on either side of joint 300. Inside radius 342 is visible in other figures of this document, including FIG. 1. As another example, a tension surface can be seen at edge 348 of aperture 410 in pendent 400. A tensile force exerted on one or more of these tension surfaces can propel device 50 in the direction shown at arrow 340.

In addition, device 50 can be manipulated by grasping pendent 400 on a face or on an edge using a suitable clamping tool having a jaw, such as a forceps.

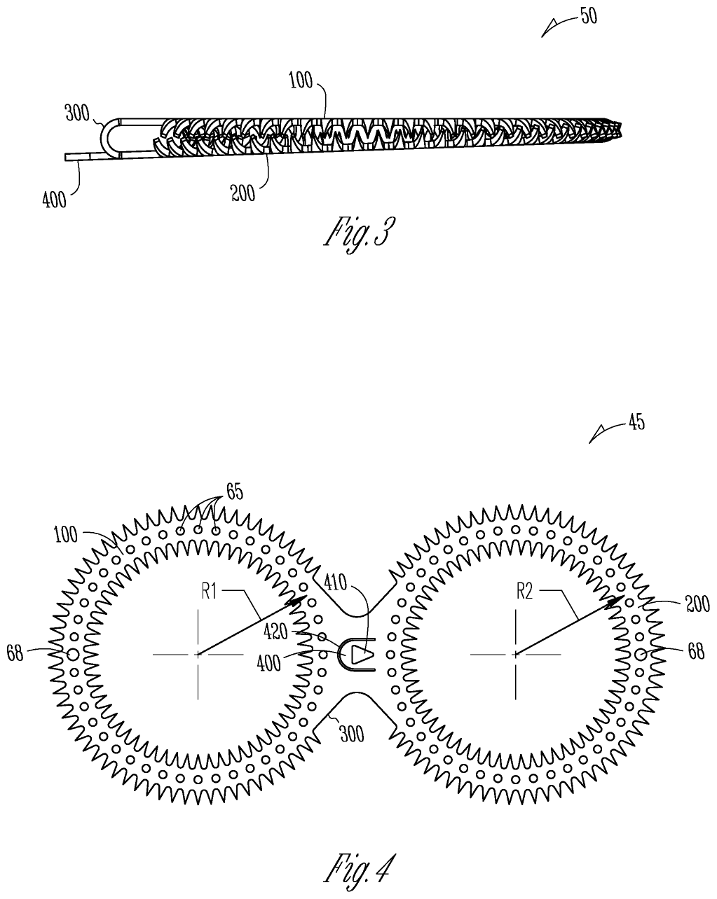

FIG. 3 illustrates a view of device 50, according to one example. In the figure, pendent 400 can be seen to extend from second band 200. The plurality of teeth disposed about the edge of first band 100 are shown to interleave with the plurality of teeth disposed about the edge of second band 200.

FIG. 4 illustrates a view of blank 45 which can be formed to produce device 50 as shown elsewhere in this document. Blank 45 has a substantially planer configuration and in later processing, blank 45 can be folded in the manner of an open hem.

In the figure, blank 45 includes first band 100 (shown on the left) and second band 200 (shown on the right) and coupled by joint 300. First band 100 has a nominal diameter corresponding to radius R1 and second band 200 has a nominal diameter corresponding to radius R2. In various example, R1 and R2 can be substantially equal or can be of different dimensions.

In the example shown, aperture 68 is indicated in segments of the rings distal to joint 300 and a plurality of band holes 65 are distributed about first band 100 and second band 200. Pendent 400 is bound by curved slot 420 disposed proximate first band 100 and coupled to the ring of second band 200. Pendent 400 includes aperture 410 having a polygon shape in the example shown.

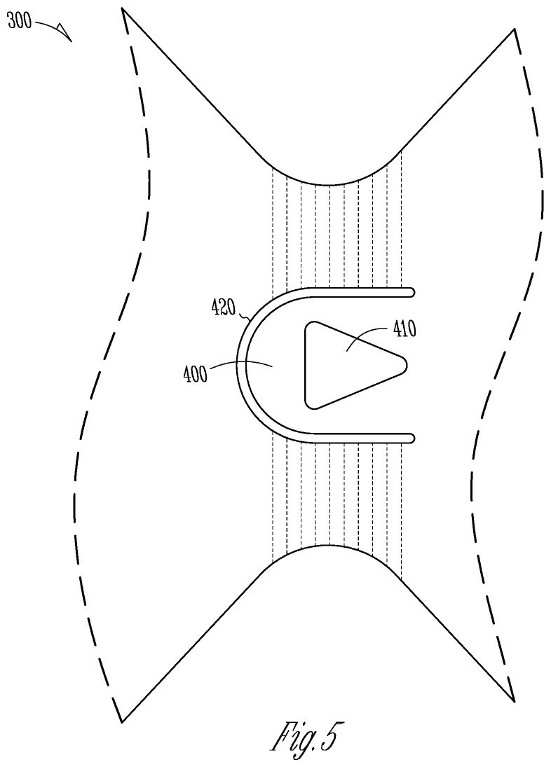

FIG. 5 illustrates an enlarged view of joint 300. In the figure, curved slot 420 partially encompasses pendent 400 and allows pendent 400 to remain in line with second band 200 while other portions of joint 300, along with first band 100, are folded in the manner described herein. Through-hole 410 in pendent 400 provides surfaces to engage and manipulate device 50.

The parallel broken lines in the figure illustrate a region of bending in the process of fabrication. Pendent 400 remains unbent and portions both above and below curved slot 420 are formed as shown in the figures.

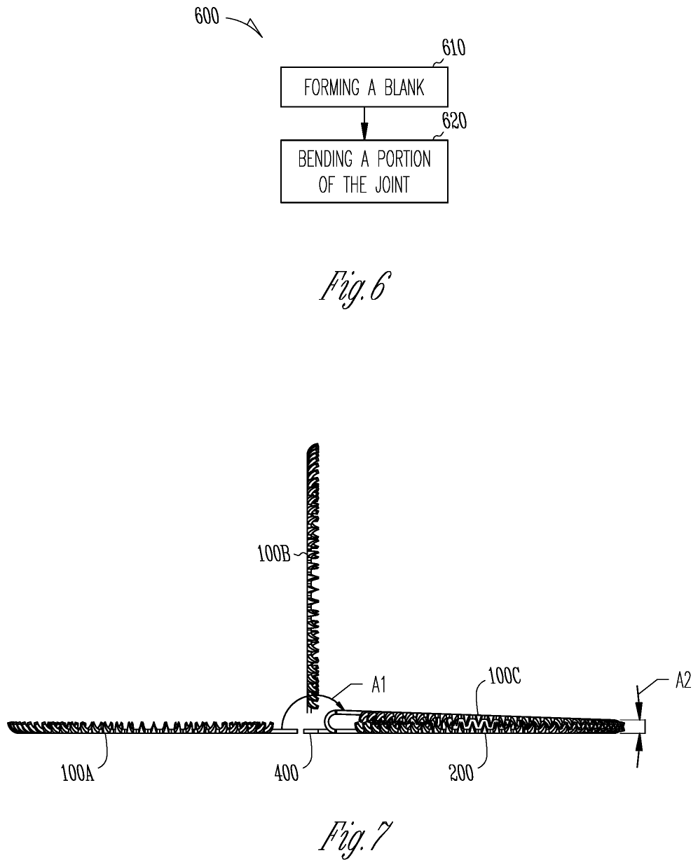

FIG. 6 illustrates a flow chart of method 600 for fabricating device 50. At 610, method 600 includes forming a blank, such as blank 45 as shown in FIG. 4. Forming a blank can include cutting or punching a sheet stock in the manner as shown in FIG. 4. Cutting can include die cutting or etching or other machining operation. The sheet stock can include a biocompatible metal, a foil, or a synthetic material.

At 620, method 600 includes bending a portion of the joint. With reference to FIG. 5, bending can include forming a radiused curve at the narrowed portion and through a portion of slot 420, in the region denoted by parallel broken lines. Bending, in the manner shown in the figures, entails forming an open hem. In an open hem, a gap remains between interior surfaces of first band 100 and second band 200.

As part of fabrication, some examples of the present subject matter are thermally processed to impart a shape memory quality to finished device 50.

As part of fabrication, some examples of the present subject matter are electropolished, coated, or finished with a particular surface profile.

One aspect of the present subject matter includes a method for forming a blank and bending a portion of the joint. Forming a blank can include forming a first ring, a second ring, and a joint coupling the first ring and the second ring. The blank can have a substantially planer form. The joint can include a first through-hole disposed in a pendent segment. While bending the portion of the joint, the pending segment is stabilized to prevent deformation and bending can include bringing the first ring proximate the second ring. Forming the blank can include cutting sheet stock. The method can also include electropolishing. In one example, bending includes forming an open hem as between the first ring and the second ring. Forming the open hem can include deflecting the rings at an angle greater than 180 degrees. In one example, bending includes forming an acute angle between a plane of the first ring and a plane of the second ring. Forming the acute angle can include forming an angle less than 10 degrees. One example includes setting a plurality of teeth at an edge of at least one of the first ring and the second ring. In one example, the method includes positioning at least one tooth of the plurality of teeth of the first ring between adjacent teeth of a plurality of teeth of the second ring. In one example, the method includes thermally treating the blank.

FIG. 7 illustrates an example of a forming process, according to one example. In the figure, second band 200 remains stationary and first band 100 is rolled, or folded. In an initial configuration (depicted at first band 100A), first band 100A is substantially planar as shown in FIG. 4). At a time during forming, first band 100B is substantially orthogonal relative to second band 200. At a later time, first band 100C is shown to have been manipulated through a rotation having angle A1. Angle A1 can be described as overbent in the sense that a plane of first band 100 passes through an angle greater than 180 degrees. In one example, angle A2 is an acute angle of approximately 10 degrees or less.

### Various Notes

In one example, pendent 400 is coupled to second band 200. In one example, pendent 400 is coupled to first band 100.

In one example, device 50 is fabricated of a biocompatible material, or a material inert to immune system, or inert to electrophysiology. Suitable materials can include nitinol, stainless steel, and drug-coated materials selected to inhibit inflammatory response (or to minimize or reduce neurological dysfunction). One example includes a polymer or a polymer combination.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A device comprising:
a first band configured as a first ring of a sheet stock;
a second band configured as a second ring of the sheet stock; and
a joint coupling the first band and the second band in stacked alignment and having a gap therebetween, the joint having a thrust surface configured to receive an applied compressive force having a direction lying substantially within a plane corresponding to at least one of the first ring or the second ring and the joint having a tension surface configured to receive an applied tensile force substantially aligned on the direction, the tension surface disposed at a pendent segment of the sheet stock and coupled to the first ring or the second ring, the pendent segment having a polygon shaped aperture to enable controlled rotation about a center axis normal to the sheet stock of the pendent segment and wherein an edge of the aperture provides the thrust surface or the tension surface, and further wherein the joint is compliant about a rotary axis lying in the plane and wherein the joint is substantially non-compliant about an axis out of the plane, and wherein the polygon shaped aperture enables control of the position and orientation of the first band, the second band, and the joint.

2. The device of claim 1 wherein at least one of the thrust surface and the tension surface includes a radiused surface.

3. The device of claim 1 wherein at least one of the thrust surface and the tension surface includes a flat surface.

4. The device of claim 1 wherein the thrust surface is disposed at a void on a portion of the first band.

5. The device of claim 4 wherein the tension surface is coupled to the second band.

6. The device of claim 5 wherein the aperture includes a portion of a through-hole.

7. (canceled)

8. The device of claim 1 wherein a plane of the first ring differs from a plane of the second ring.

9. The device of claim 1 wherein at least one of the first band and the second band includes a transverse aperture disposed radially opposite a location of the joint.

10. The device of claim 1 wherein at least one of the first band and the second band includes a sawtooth edge.

11. The device of claim 10 wherein the sawtooth edge is set towards the gap.

12. The device of claim 1 wherein the first band includes a first sawtooth edge and the second band includes a second sawtooth edge and wherein a first tooth of the first sawtooth edge is aligned between an adjacent pair of teeth of the second sawtooth edge.

13. The device of claim 1 wherein at least one of the first band and the second band includes an aperture.

14. The device of claim 1 wherein the first band, the second band, and the joint include a shape memory material.

15. The device of claim 14 wherein the shape memory material includes a metal alloy.

16. The device of claim 15 wherein the metal alloy includes nitinol.

17. The device of claim 1 wherein the first band, the second band, and the joint are integrally formed.

18. The device of claim 1 wherein the joint includes a live hinge.

19. The device of claim 1 wherein a first diameter of the first ring and a second diameter of the second ring are unequal.

20. A blank for forming a tissue scaffold, the blank comprising:
a first ring of a sheet stock;
a second ring of the sheet stock; and
a joint coupling the first ring and the second ring, wherein the joint, the first ring, and the second ring have a planar form, and wherein the joint includes a first through-hole disposed in a pendent segment of the sheet stock, and wherein the first through-hole has a polygon shape configured to enable controlled rotation about a center axis normal to the sheet stock of the pendent segment and wherein an edge of the first through-hole provides a thrust surface or a tension surface, and further wherein the joint is substantially non-compliant about an axis out of the plane, and wherein the first through-hole enables control of the position and orientation of the first ring, the second ring, and the joint.

21. The blank of claim 20 wherein the joint includes nitinol.

22. (canceled)

23. The blank of claim 20 wherein the polygon shape includes a triangle.

24. The blank of claim 20 wherein the pendent segment is bounded by a curved slot.

25. The blank of claim 20 wherein at least one of the first ring and the second ring includes a sawtooth edge.

26. A method comprising:
forming a blank of a sheet stock having a first ring, a second ring, and a joint coupling the first ring and the second ring, wherein the blank has a substantially planar form, and wherein the joint includes a first through-hole disposed in a pendent segment of the sheet stock, wherein the first through-hole has a polygon shape configured to enable controlled rotation about a center axis normal to the sheet stock of the pendent segment and wherein an edge of the first through-hole provides a thrust surface or a tension surface; and
bending a portion of the joint while preventing deformation of the pendent segment to bring the first ring proximate the second ring and wherein the through-hole enables control of the position and orientation of the first ring, the second ring, and the joint, and further wherein the joint is compliant about a rotary axis lying in the plane and wherein the joint is substantially non-compliant about an axis out of the plane.

27. The method of claim 26 wherein forming includes cutting the sheet stock.

28. The method of claim 26 further including electropolishing.

29. The method of claim 26 wherein bending includes forming an open hem.

30. The method of claim 29 wherein forming the open hem includes deflecting greater than 180 degrees.
